(19) United States
(12) Patent Application Publication
(10) Pub. No.: US 2018/0318534 A1
(43) Pub. Date: Nov. 8, 2018

(54) ENDOTRACHEAL INTUBATION DEVICE

(71) Applicants: Peter Desatnik, Lund (SE); Mats Christensson, Staffanstorp (SE)

(72) Inventors: Peter Desatnik, Lund (SE); Mats Christensson, Staffanstorp (SE)

(21) Appl. No.: 15/773,648

(22) PCT Filed: Oct. 20, 2016
(86) PCT No.: PCT/EP2016/075212

§ 371 Date: May 4, 2018

(30) Foreign Application Priority Data
Nov. 6, 2015 (SE) 1551439-1

(51) Int. Cl. A61M 16/04

(52) U.S. Cl. CPC A61M 16/0418; A61M 16/0488; A61M 16/0434; A61M 16/0411

(57) ABSTRACT

The present disclosure relates to an endotracheal intubation device (2, 3) comprising: a hollow endotracheal tube (2) having a proximal portion (4); a distal portion (5); and a flexible portion (6) located between the proximal portion and the distal portion, the flexible portion being more flexible than the proximal portion and the distal portion; and an intubation stylet (3) having a proximal part (10); a distal part (11); a bendable part (12) located between the proximal part and the distal part, configured for slidable insertion into an engagement position in the hollow endotracheal tube; and a flexion mechanism (13-18) configured to control deflection of the bendable part, the endotracheal intubation device configured such that the flexible portion of the endotracheal tube is substantially aligned with the bendable part of the intubation stylet in said engagement position. The disclosure further relates to an intubation stylet having a proximal part; a distal part; a bendable part located between the proximal part and the distal part, configured for slidable insertion into an engagement position in the hollow endotracheal tube; and a flexion mechanism configured to control deflection of the bendable part.

The present disclosure relates to an endotracheal intubation device having a hollow endotracheal tube and an intubation stylet. The disclosure further relates to a bendable intubation stylet for providing a conduit to the trachea of a patient.

### BACKGROUND OF INVENTION

Endotracheal tubes are used in a variety of medical situations to provide a conduit to the trachea of a patient. A tracheal tube is a catheter that is inserted to the trachea for establishing an airway. Typically, the purpose is to ensure an exchange of oxygen and carbon dioxide. More specifically, an endotracheal tube is inserted through the mouth (orotracheal) or through the nose (nasotracheal) into the trachea. Most endotracheal tubes are constructed of plastic polymers or rubber. In some cases the endotracheal tubes have an inflatable cuff to seal the trachea against air leakage and aspiration of gastric contents, blood, secretions and other fluids.

The placement of an endotracheal tube into the trachea of a patient may be a difficult procedure due to an anatomically challenging introduction path, individual anatomic differences between patients, limited visibility etc. When inserted through the throat of the patient, the endotracheal tube should pass through the glottis opening further on to the trachea. In the human body, the epiglottis is positioned over the glottis opening to prevent that ingested material enters the trachea. Obviously, the endotracheal tube must not enter the esophagus in endotracheal intubation of a patient. Nevertheless, it is not uncommon that esophageal intubation is unintentionally performed instead of tracheal intubation.

An intubation stylet is sometimes used to facilitate difficult intubation. The purpose of using an intubation stylet is to assist tracheal intubation by stiffening the tube as it is inserted into the throat of the patient. As stated, endotracheal tube is typically made of plastic polymers or rubber and may kink, curl or bend if placed without using a stiffer intubation stylet during insertion. Once the endotracheal tube is in place, the intubations stylet can be removed. As stated, introducing the endotracheal tube, with or without an intubation stylet, is often difficult since the section of the throat above the glottis opening is curved. There are examples of slightly bendable intubation stylets in the art; however, these stylets are inconvenient in terms of operation and access and insertion to the trachea.

### SUMMARY OF INVENTION

The present disclosure relates to improvements of existing devices for performing endotracheal intubation. More specifically, the disclosure relates to an endotracheal intubation device having a hollow endotracheal tube and an intubation stylet having advantages in relation to the operation of the device. In a preferred embodiment the presently disclosed endotracheal intubation device comprises: a hollow endotracheal tube having a proximal portion; a distal portion; and a flexible portion located between the proximal portion and the distal portion, the flexible portion being more flexible than the proximal portion and the distal portion; and an intubation stylet having a proximal part; a distal part; a bendable part located between the proximal part and the distal part, configured for slidable insertion into an engagement position in the hollow endotracheal tube; and a flexion mechanism configured to control deflection of the bendable part, the endotracheal intubation device configured such that the flexible portion of the endotracheal tube is substantially aligned with the bendable part of the intubation stylet in said engagement position. By aligning the bendable part of the intubation stylet and flexible portion (being more flexible than the proximal portion and distal portions) of the of the endotracheal tube, an endotracheal intubation device is obtained, which is more easily inserted into the trachea while a substantial part of the endotracheal tube can still be relatively rigid in order not to kink, curl or bend. One advantage of such a solution is that the bendable part is more easily controlled while inserted into the trachea. Typically a laryngoscope is used as assistance during intubation. With a device that is more easily handled, the need for visibility may be reduced and therefore also the insertion of the laryngoscope may be less painful for the patient.

A flexion mechanism is configured to control deflection of the bendable part of the intubation stylet—therefore the bendable part is preferably configurable in at least an unflexed state and a flexed state. In relation to the insertion of the endotracheal intubation device into the trachea, the endotracheal intubation device may be inserted into the mouth or nose of the patient, wherein the distal end is initially positioned adjacent to the glottis opening. As stated, the section of the throat above the glottis opening is typically curved and therefore there is a risk the device accidently enters the esophagus if inserted further into the throat. In the position near the glottis opening, the bendable part may be changed to a flexed state. The proximal part and the distal part of the intubation stylet may thereby form an angle that is suitable for inserting the distal end of the device through the glottis opening into the trachea. Preferably, the shape of the bendable part corresponds substantially to the shape of the throat just above the glottis opening. The bendable part is preferably easily adjustable and/or controllable in order to enable the user to insert the device in a controlled manner, for example by visually observing the device while inserted and deflecting the bendable part of the intubation stylet. The flexible portion of the hollow endotracheal tube plays a role in that it provides a section that makes that device easily operated while the endotracheal tube as a whole may still be sufficiently stable for being used in a second operating mode for maintaining an open airway of the patient.

The disclosure further relates to an intubation stylet having a proximal part; a distal part; a bendable part located between the proximal part and the distal part, configured for slidable insertion into an engagement position in the hollow endotracheal tube; and a flexion mechanism configured to control deflection of the bendable part. The intubation stylet may comprise a handle member, preferably a single-handed handle member, in connection with a pulling mechanism configured to pull a wire attached to the distal part of the intubation stylet, the wire being further attached to the flexion mechanism and/or a pulling mechanism. In a preferred embodiment, the intubation stylet comprises a pressure element configured to be operated by the thumb of a user holding the handle member with the index-, middle-, ring- and little fingers, preferably by having the index-, middle-, ring- and little fingers are placed around the hollow endotracheal tube, preferably with the little finger towards the distal end of the endotracheal tube. Such a grip has the advantage that the thumb can regulate the deflection while the rest of the fingers control the position of the device. The device can thus be entirely operated by one hand. One way of using the device in an intubation process involves holding the device as explained in one hand and a laryngoscope in the other hand. The bendable part of the endotracheal intubation device may comprise a substantially flat section. This has the advantage over for example a coiled spring that the bendable part is only bendable in a predetermined bending direction and substantially rigid sideways in relation to the predetermined bending direction. Preferably, the substantially flat section is straight in relation to the general curve of the intubation device in the unflexed state. This means that for a lateral view of the device, as for example in FIG. 8b, there is a general bending of the device in an XY plane, and the substantially flat section is then substantially straight in relation to the rest of the intubation device and/or bendable part of the intubation device, which prevents that the bendable part is accidently flexed during insertion.

The bendable part of the intubation stylet may be elastic in the sense that it has a tendency to flex back to the initial unflexed state. In one embodiment, this is realized by means of a flexion mechanism to control deflection of the bendable part, wherein moving a part of the flexion mechanism in one direction using a force corresponds to a deflecting movement of the bendable part, for example by pushing a pressure element, and wherein releasing the force causes the bendable part to move back to the unflexed state. The insertion of the device is thereby operated by pushing an element to flex the bendable part and releasing to unflex the bendable part. This may be achieved by e.g. a coiled spring being used as the bendable portion of the intubation stylet, wherein the intubation stylet can be described as having inherent potential energy in the flexed state.

In one embodiment, the aligned flexible portion of the endotracheal tube and bendable part of the intubation stylet constitute a limited section of the device near the distal end of the device. One advantage of such an embodiment is that only the section of the device flexes while the rest of the device is substantially rigid as long as the intubation stylet is maintained in the aligned position inside the endotracheal tube in the first operating mode, wherein the device is configured to be inserted into the trachea of a patient.

These and other aspects of the invention are set forth in the following detailed description if the invention.

### DESCRIPTION OF DRAWINGS

FIG. 1a shows an embodiment of the presently disclosed intubation stylet in an unflexed state.

FIG. 1b shows the intubation stylet of FIG. 1 in a flexed state.

FIG. 2 shows a further embodiment of the presently disclosed intubation stylet.

FIG. 3 shows an embodiment of the presently disclosed hollow endotracheal tube.

FIG. 4 shows an embodiment of the presently disclosed endotracheal intubation device having a hollow endotracheal tube and an intubation stylet, the parts presented side by side.

FIG. 5 shows the endotracheal intubation device of FIG. 4, wherein the intubation stylet is in a flexed state.

FIG. 6 shows the endotracheal intubation device of FIG. 4 and FIG. 5, wherein the intubation stylet is slidably inserted into an engagement position in the hollow endotracheal tube, the flexible portion of the hollow endotracheal tube being substantially aligned with the bendable part of the intubation stylet.

FIG. 7 shows an embodiment of an intubation stylet, wherein the proximal part comprises a most proximal part which has a larger diameter than the rest of the intubation stylet and is substantially straight.

FIG. 8 shows an embodiment of the distal and bendable parts of an intubation stylet, wherein the bendable part has a section of the bendable part that is substantially flat, and the section is straight in relation to the general curve of the intubation device in the unflexed state.

### DETAILED DESCRIPTION OF THE INVENTION

The present disclosure relates to an endotracheal intubation device having a hollow endotracheal tube and an intubation stylet having advantages in relation to the operation of the device. In a preferred embodiment the presently disclosed endotracheal intubation device comprises: a hollow endotracheal tube having a proximal portion; a distal portion; and a flexible portion located between the proximal portion and the distal portion, the flexible portion being more flexible than the proximal portion and the distal portion; and an intubation stylet having a proximal part; a distal part; a bendable part located between the proximal part and the distal part, configured for slidable insertion into an engagement position in the hollow endotracheal tube; and a flexion mechanism configured to control deflection of the bendable part, the endotracheal intubation device configured such that the flexible portion of the endotracheal tube is substantially aligned with the bendable part of the intubation stylet in said engagement position.

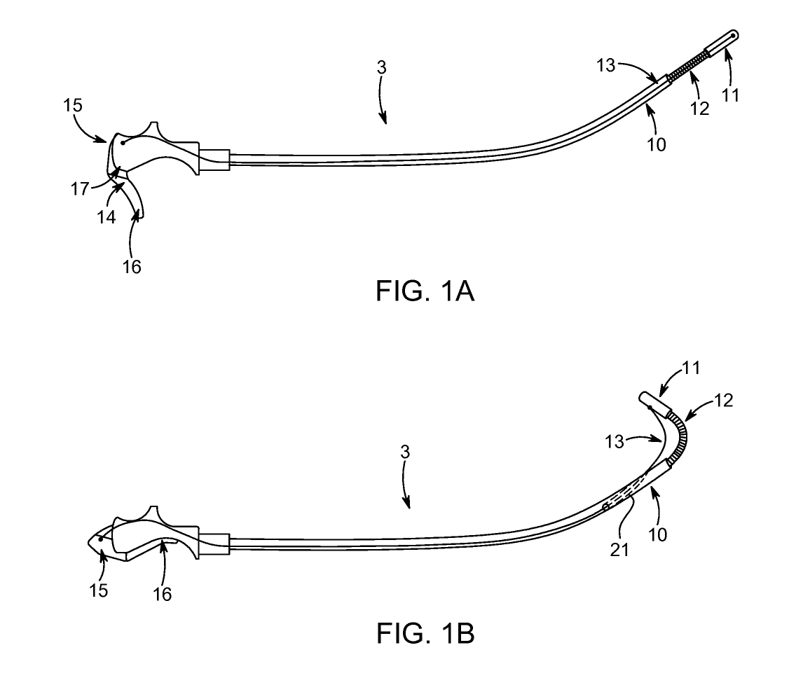

As stated, the alignment of the bendable part of the intubation stylet and flexible portion (being more flexible than the proximal portion and distal portions) of the endotracheal tube provides a design which as beneficial for the introduction and operation of the device. FIGS. 1a and 1b is an illustrative example of how the presently disclosed endotracheal intubation device may be used. The device is typically inserted in a first operating mode, wherein the device is configured to be inserted into the trachea of a patient, the intubation stylet positioned inside the endotracheal tube, the flexible portion of the endotracheal tube being substantially aligned with the bendable part of the intubation stylet. The device may be inserted in the position showed in FIG. 1a. In this example the proximal part 10 of the intubation stylet is slightly curved and the bendable part 12 and distal part 11 form a substantially straight shape while the device approaches the critical area near the glottis opening. In order to be inserted into the trachea through the glottis opening it may now be useful to bend the intubation stylet (and thereby the whole device), preferably only a limited section of the intubation stylet as shown in FIG. 1b, wherein the bendable part is bent to be inserted into the trachea more easily.

The bending and insertion may be performed in a controlled manner since the bendable part of the intubation stylet is substantially aligned with a flexible portion of the endotracheal tube. This has the advantage that the section which is bent can be relatively easily bent while the endotracheal tube as a whole is still sufficiently rigid to be used in a second operating mode without the intubation stylet for maintaining an open airway of the patient. If only a limited section of the endotracheal tube (the flexible portion) is made more flexible than the rest of the tube, the tube will stand better chances against collapsing, kinking, curling or bending during use as an airway path. A conventional tube may typically also be described as slightly flexible; however a minimum of rigidity is needed in order not to kink, curl or bend. The presently disclosed endotracheal tube differs from conventional tubes in that it has a section that is more flexible than the proximal and distal sections, which is described in further detail below.

The presently disclosed endotracheal intubation device may be used for intubation of a human patient. Therefore, the device may have a length of between 100 mm and 600 mm, or between 200 mm and 400 mm, or between 200 mm and 300 mm, or between 50 mm and 200 mm, or between 100 mm and 500 mm, or between 100 mm and 300 mm, or longer than 200 mm.

Intubation Stylet

As stated, the intubation according to the present disclosure has a proximal part; a distal part; a bendable part located between the proximal part and the distal part. The intubation stylet is configured for slidable insertion into an engagement position in the hollow endotracheal tube. In one embodiment, the bendable part of the intubation stylet is configurable in at least an unflexed state and a flexed state. Examples of these two states are shown in FIGS. 1a (unflexed) and 1b (flexed). Any position between the unflexed state and the flexed state is also possible to achieve by controlling the flexion mechanism of the device. In a preferred embodiment, the deflection is controlled such that any position between the flexed state and the unflexed state can be achieved.

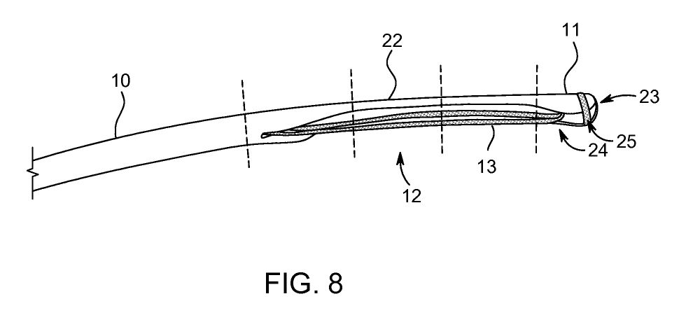

The bendable part of the endotracheal intubation device may comprise a substantially flat section. This has the advantage over for example a coiled spring that the bendable part is only bendable in a predetermined bending direction and substantially rigid sideways in relation to the predetermined bending direction. This design of the bendable part can be seen in FIGS. 7-8. In the example of FIG. 8 it can be seen that the bendable part (12) has a section (22) of the bendable part that is substantially flat.

The substantially flat section may have a thickness of less than 5 mm, preferably a thickness of less than 4 mm, even more preferably a thickness of less than 3 mm, most preferably a thickness of less than 2 mm. By having a flat design that is relatively thin in relation to the rest of the intubation stylet, a bending part which is both flexible in the plane that follows the general two-dimensional bending curve of the device (XY) and rigid in the Z direction is obtained. Preferably the design also has an inherent capability of flexing back from the flexed state to the unflexed state automatically.

The substantially flat section is preferably straight in relation to the general curve of the intubation device in the unflexed state. This means that for a lateral view of the device, as for example in FIG. 8b, there is a general bending of the device in an XY plane, and the substantially flat section is then substantially straight in relation to the rest of the intubation device and/or bendable part of the intubation device, which prevents that the bendable part is accidently flexed during insertion.

The substantially flat section having a length of 5-30 mm, preferably 5-20 mm, more preferably 5-15 mm, even more preferably 8-12 mm. There may be a smooth transition from the rest of the intubation device, which typically has a circular cross-section, and the substantially flat section.

The degree of deflection may be expressed as an angle between the proximal part and the distal part of the intubation stylet. Since the bendable part is limited to a middle section between the proximal part and the distal part, it is possible to achieve a relatively high deflection corresponding to the curved part of the throat above the glottis opening of the patient. An illustration of how to construe the angle is shown in FIG. 5 (angle 19). The angle between the proximal part and the distal part of the intubation stylet may be between 50° and 179°, or between 60° and 179°, or between 70° and 179°, or between 80° and 179°, or between 60° and 160°, or between 60° and 150°, or between 60° and 140°, or between 40° and 130°, or between 60° and 120°, or between 80° and 100°, or between 70° and 110°, such as 50°, or 55°, or 60°, or 65°, or 70°, or 75°, or 80°, or 85°, or 90°, or 95°, or 100°, or 105°, or 110°, or 115°, or 120°, or 125°, or 130°, or 135°, in the flexed state. As explained, typically any position between the flexed state and the unflexed state are also possible.

In one embodiment the proximal part of the intubation comprises a most proximal part having an outer diameter that is larger than the rest of the proximal part and/or the rest of the intubation device. By having a most proximal part having a larger outer diameter, the stylet is stiffer in the most proximal part, which may be an advantage during insertion into the trachea of the patient. In this embodiment the stylet may have a least three levels of rigidity. The most proximal part is the most rigid. An intermediate part corresponding to a section comprising to the rest of the proximal part is less rigid but still having some rigidity. The bendable part is less rigid and configured to be flexed during insertion into the trachea. The most proximal part may be substantially straight and rigid.

The most proximal part may have an outer diameter that is at least 0.3 mm larger than the rest of the proximal part, preferably an outer diameter at least 0.5 mm larger than the rest of the proximal part, even more preferably an outer diameter at least 0.7 mm larger than the rest of the proximal part, most preferably an outer diameter at least 1.0 mm larger than the rest of the proximal part. The length of the most proximal part mahy be between 50 mm and 150 mm, preferably between 70 mm and 130 mm, even more preferably between 80 mm and 120 mm.

Furthermore, the bendable part of the intubation stylet may be elastic. Elasticity is the ability of a body to resist a distorting influence or stress and to return to its original size and shape when the stress is removed. For the presently disclosed intubation device, elastic in this sense shall be construed as the ability of the bendable part to return to its original state, typically the unflexed state, without a need for the user to apply a force. In practice this means that the user will apply a force to bend the bendable part of the intubation device into a flexed state, and when the force is released, the bendable part will return to the unflexed state. The force can be applied and released gradually to adjust the shape of the intubation stylet such that it can be introduced into the trachea. The elasticity may be achieved by the substantially flat section of the bendable part of the intubation stylet.

Preferably, the elasticity is responsive with no, or substantially no, or very little delay in the sense that when the force by the user is released, the user will experience that the bendable part starts returning to its original position immediately. The inventors have realized that a coiled spring may represent a suitable embodiment for this purpose. Therefore, in one embodiment, the bendable part of the intubation stylet comprises a coiled spring. An example of such an embodiment is shown in FIG. 1 (the bendable part 12 being a coiled spring). In another embodiment, the elastic effect is achieved by a thinner portion of the intubation device. The relatively thin part is preferably made of an elastic material, which returns to the unflexed state as a default position. In yet another embodiment, the bendable part may be a portion that is relatively thin and/or at least thinner than the proximal and distal parts, the relative thin portion being toothed. Alternatively the relatively thin portion could be reinforced with a second material further to the main material of the relatively thin portion part. A further embodiment of the bendable part may be a combination of a relatively thin portion and a spring coil, wherein the thin portion would be easily bendable and the spring could primarily having the function of pulling the intubation stylet back to its original position. In one embodiment, the intubation stylet may also be seen as a stick or rod, wherein the relatively thin portion corresponds to a section of the intubation stylet having been cut away, grinded or otherwise removed from the stick or rod.

Preferably, the material is, despite its elasticity, sufficiently rigid to be safely introduced into the trachea without changing its shape except for the bending caused by the user. In one embodiment, the bendable part of the intubation stylet is thinner than the proximal part and the distal part of the intubation stylet. The elasticity in the context of the bendable part of the intubation stylet may be expressed as an inherent potential energy of the bendable part in the flexed state. In one embodiment, the bendable part of the intubation stylet has inherent potential energy in the flexed state, which is released when the user releases the force on the trigger or handle of the device. When the inherent potential energy is released, the bendable part returns to the starting position, typically the unflexed state.

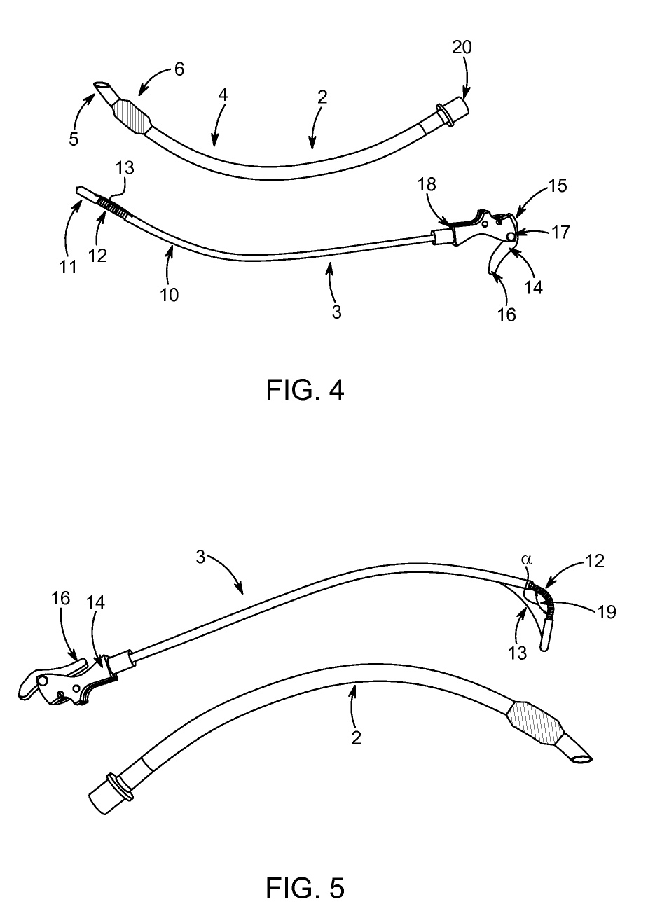

Furthermore, the intubation stylet may be slightly curved, conforming to an upper airway of the patient, and relatively stiff. An example of a curved intubation stylet is shown in FIG. 4. The purpose of the curved shape is the combination of the curved shape and the even more curved shape of the bendable part is the provision of a device having a section fitting to the part of the throat substantially in level with the pharynx above the trachea and larynx and a (usually shorter) bendable section (bendable part), the latter being directly controlled by a user. The shape of the intubation in the unflexed configuration stylet preferably matches the shape of the endotracheal tube.

The intubation stylet may be made of a substantially rigid material, such as ceramics, ceramic composites, metal, such as aluminium or steel, or plastics, such as polycarbonate (PC) or polymethylmethacrylate (PMMA), or any other substantially rigid material suitable for the purpose. The intubation stylet may also be made of a polyamide elastomer. In one embodiment, for example having an intubation device entirely made of a polyamide elastomer, the intubation device is non-magnetic. In this embodiment the device does not influence electromagnetic devices. Furthermore the material of the intubation stylet is preferably a low-friction material for smooth insertion of the intubation device into the endotracheal tube.

Suitable sizes of the intubation stylet include the intubation stylet having a stylet outer diameter between 1 mm and 15 mm, or between 2 mm and 15 mm, or between 1 mm and 10 mm, or between 2 mm and 10 mm, such as 1 mm, or 2 mm, or 3 mm, or 4 mm, or 5 mm, or 6 mm, or 7 mm, or 8 mm, or 9 mm, or 10 mm, or 11 mm, or 12 mm, or 13 mm, or 14 mm, or 15 mm. Logically, the outer diameter of the intubation is smaller than the inner diameter of the endotracheal tube.

Pulling Mechanism and Wire

One aspect of the presently disclosed intubation device relates to the flexion mechanism comprising a pulling mechanism extending from a proximal end of the intubation stylet to the distal part of the intubation stylet. In one embodiment, the pulling mechanism exerts a pulling force on the distal part of the intubation stylet in the flexed state. In this embodiment the unflexed state can be said to correspond to a released state wherein the pulling mechanism does not pull the distal part of the intubation stylet. This is the situation shown in FIG. 1a. When the pulling mechanisms pulls the distal part, as shown in FIG. 1b, the bendable part of the intubation stylet bends accordingly. As explained, the intubation stylet may be considered to possess inherent potential energy in the flexed state. When the pulling force is released, the inherent potential energy causes the intubation stylet to return to the unflexed state providing any additional force.

In a preferred embodiment, the flexion mechanism comprises a wire attached to the distal part of the intubation stylet. In one embodiment, the wire is attached to the distal part of the intubation stylet in one end and to the pulling mechanism in the other end. The inventors have realized that the wire operates more efficiently if it operates from a point closer to the bendable part of the intubation stylet. Furthermore, if the wire would be directly connected between the distal part of the intubation stylet and the pulling mechanism located in the proximal end of the device, the wire would not run along the intubation stylet, rather would the wire, the proximal part and the bendable part with the distal part form a triangle, which would be inconvenient in the operation of the device. For this reason, in one embodiment the wire extending from the proximal part of the intubation stylet to the distal part of the intubation stylet may be arranged through a fixed point at the intubation stylet and/or through a duct of the intubation stylet. This feature is illustrated in FIG. 1. In FIG. 1b it can be seen that the wire runs on the outer side of the intubation stylet. Near the bendable part the wire is guided through a duct in the intubation stylet, where the duct serves as a point around which the bendable part can be bent.

In one embodiment the wire is a double wire. The double wire is preferably the same wire formed as a loop in the distal end. A double wire is a more secure way of providing the pulling mechanism between the proximal and distal part of the intubation device.

Furthermore it may distribute the pulling force more evenly over a flat section of the bendable part to avoid that bending occurs in the Z plane (i.e. in any direction that is not in the general curve of the device in the XY plane.)

Preferably the double wire is attached to a distal tip of the distal end of the intubation device. This can be achieved by a loop formed by the double wire, wherein the loop is attached around the tip and for example placed in a slot extending around the distal tip of the intubation device, as shown in FIG. 8. The intubation stylet may also comprise a through-hole (24 in FIG. 8) adjacent to a distal tip of the intubation stylet, wherein the wire is configured to run through the through the through-hole and attach around the tip.

Handle Member

As stated, the present disclosure relates not only to the complete intubation device, but also to an intubation stylet having a proximal part; a distal part; a bendable part located between the proximal part and the distal part, configured for slidable insertion into an engagement position in the hollow endotracheal tube; and a flexion mechanism configured to control deflection of the bendable part. Preferably, the flexion mechanism comprises a pulling mechanism extending from a proximal end of the intubation stylet to the distal part of the intubation stylet. The flexion mechanism may also comprise a wire attached to the distal part of the intubation stylet, the wire being further attached to the flexion mechanism and/or pulling mechanism. In an emergency situation, or simply in any situation in which intubation has to be performed, there is a risk that medical staff, e.g. due to lack of skills, inexperience or stress, loses valuable time if the procedure fails one or several times. In many such situations it may also be valuable to be able to operate the intubation stylet with one hand. Therefore, in one embodiment, the intubation stylet further comprises a handle member, preferably a single-handed handle member, in connection with the pulling mechanism. A single-handed handle member may be combined with any of the abovementioned intubation stylet features. A particularly useful combination is a single-handed handle member and an elastic bendable part as described above since this embodiment enables truly single-handled control of the intubation stylet. By pushing a pressure element connected to the wire to flex the bendable part and releasing the same pressure element to unflex the bendable, the user may control the device using one hand. In a preferred embodiment, the pressure element is configured to be operated by the thumb of a user holding the handle member with the index-, middle-, ring- and little fingers, preferably by having the index-, middle-, ring- and little fingers placed around the intubation stylet or handle, preferably with the little finger towards the distal end of the intubation stylet. In one embodiment the handle member is detachable, which means that the device is configurable in the sense that different sizes and lengths of the endotracheal tube and intubation stylet can be connected to the handle member, depending on the patient.

In one embodiment, a lever is used to operate and control the wire attached to the distal part of the intubation device. In one embodiment the other end of the wire is attached to the flexion mechanism and/or lever. The mechanism may be arranged such that when a pressure element connected to the lever is pushed, the flexion mechanism pulls the wire and bends the bendable part of the intubation stylet. In one embodiment, the lever comprises a rigid rod pivotable at a fixed hinge, wherein the lever is configured to pull the wire when the pressure element is pushed. An example of such a solution is shown in FIG. 1a and FIG. 1b. The lever 14 has a proximal rod element 15 and a distal rod element 16, which are pivotable at the fixed hinge 17. In FIG. 1a the intubation stylet is in an unflexed state. In FIG. 1b the rod element has been turned counter clockwise around the fixed hinge 17 by pushing the distal rod element in the same direction. The inventors have realized that such a mechanism is ergonomically very useful for the presently disclosed intubation device. This design may be considered to be construed under the description of the abovementioned single-handed handle.

Endotracheal Tube

The flexible portion of the hollow endotracheal tube plays a role in that it provides a section that makes that device easily operated while the endotracheal tube as a whole may still be sufficiently stable for being used in a second operating mode for maintaining an open airway of the patient. An endotracheal tube is normally flexible in order to be more easily inserted into the trachea, still they must not be easily kinked. In the presently disclosed intubation device, the endotracheal tube is constituted by three portions: the proximal portion, the distal portion and the flexible portion. In one embodiment, the proximal portion and the distal portion of the endotracheal tube are more rigid than the flexible portion of the endotracheal tube. There are several options for achieving the desired properties of the tube. In one embodiment, the middle section (flexible portion) has a thinner sidewall than the rest of the tube. In one embodiment, the flexible portion of the endotracheal tube is at least 0.1 mm, or at least 0.2 mm, or at least 0.3 mm, or at least 0.4 mm, or at least 0.5 mm, or at least 0.6 mm, or at least 0.7 mm, or at least 0.8 mm, or at least 0.9 mm, or at least 1.0 mm, or at least 1.2 mm, or at least 1.4 mm, or at least 1.6 mm, or at least 1.8 mm, or at least 2.0 mm thinner than the sidewalls of the proximal portion and the distal portion of the tube.

A further option is to use a homogenous tube as a starting point—the flexible section may then be treated e.g. by creating grooves, cracks, scratches, or any pattern rendering the structure more flexible. This can be seen as geometrical improvements of the tubular body for achieving the desired properties.

Alternative, the different sections of the tube can be made of the different materials, or some parts can be treated to obtain more rigid or more flexible portions. In one embodiment, the flexible portion is made of a softer material than the proximal and distal portions.

In one embodiment, the tube may be a conventional endotracheal tube, wherein the proximal and distal portions, but not the flexible portion, have been reinforced.

In one embodiment, the endotracheal tube is made of plastic material, such as polyvinyl chloride (PVC), or silicone plastic.

Preferably, the endotracheal tube is (slightly) curved, conforming to an upper airway of the patient.

The length of the flexible portion is preferably relatively limited. Introducing the endotracheal tube, with or without an intubation stylet, is often difficult since the section of the throat above the glottis opening is curved. Preferably the length of the flexible portion is between 10 mm and 50 mm, or between 10 mm and 40 mm, or between 10 mm and 30 mm, or between 10 mm and 20 mm, or less than 50 mm, or less than 40 mm, or less than 30 mm, or less than 20 mm. Similarly, for anatomical reasons, the flexible portion is preferably located relatively adjacent to the distal end of the device. Therefore, in one embodiment, the length of the distal portion of the endotracheal tube, and similarly since they are aligned, the distal part of the intubation stylet, is between 10 mm and 50 mm, or between 10 mm and 40 mm, or between 10 mm and 30 mm, or between 10 mm and 20 mm, or less than 50 mm, or less than 40 mm, or less than 30 mm, or less than 20 mm.

As stated, the flexible portion of the hollow endotracheal tube plays a role in that it provides a section that makes that device easily operated while the endotracheal tube as a whole may still be sufficiently stable for being used in a second operating mode for maintaining an open airway of the patient. Therefore, in one embodiment, the device is, in a second operating mode, configured to be used without the intubation stylet for maintaining an open airway of the patient. In the second configuration mode, the tube is configured such that it is not folded during use.

Suitable sizes of the hollow endotracheal tube include the tube having a device outer diameter between 5 mm and 20 mm, or between 5 mm and 15 mm, or between 5 mm and 10 mm, or between 5 mm and 10 mm, such as 5 mm, or 6 mm, or 7 mm, or 8 mm, or 9 mm, or 10 mm, or 11 mm, or 12 mm, or 13 mm, or 14 mm, or 15 mm, or 16 mm, or 17 mm, or 18 mm, or 19 mm, or 20 mm. Furthermore, the hollow endotracheal tube may be formed by a cylindrical sidewall having a thickness of less than 5 mm, or less than 4 mm, or less than 3 mm, or less than 2 mm, or less than 1 mm, or less than 0.5 mm, or less than 0.4 mm, or less than 0.3 mm, or less than 0.2 mm, or less than 0.1 mm.

Endotracheal tubes are open in both ends, enabling an air passage. Some endotracheal tubes have an additional hole at the tip called Murphy's Eye. If the main opening gets blocked, air may still flow through the Murphy eye. One embodiment of the presently disclosed invention includes at least one secondary opening at the distal portion, preferably in the cylindrical sidewall, adjacent to the tip of the endotracheal tube.

### Further Embodiments

As stated, the section of the throat above the glottis opening is typically curved and therefore there is a risk the device accidently enters the esophagus if inserted further into the throat. The area is typically not easily reached by a conventional endotracheal intubation device. In addition, it may be difficult for the person performing the intubation to see the anatomical details clearly. Therefore, as a further improvement, in one embodiment, the intubation stylet further comprising a light source at the distal end. Preferably, in this embodiment, there is also a power source, such as a battery, an electrical wire connecting the power source to the light source, and a power switch configured to activate and deactivate the light source. The ease of use may be a particularly important parameter for the device. Therefore, in one embodiment, the power switch is configured such that the light source is enabled when the bendable part is in the flexed state and disabled when the bendable part is in the unflexed state, preferably in synchronization with the flexion mechanism, such that the pressure element closes the switch when pushed.

Furthermore, the intubation device may comprise an inflatable cuff surrounding the device. Inflate cuffs are known in the art—a person skilled in the art would know how to arrange an inflatable cuff on an intubation device. Nevertheless, for the presently disclosed endotracheal intubation device, depending on the option that is chosen for the flexible portion of the endotracheal tube, it may be useful to have an inflatable cuff that covers the flexible portion of the endotracheal tube.

### DETAILED DESCRIPTION OF THE DRAWINGS

The invention will in the following be described in greater detail with reference to the accompanying drawings. The drawings are exemplary and are intended to illustrate some of the features of the presently disclosed endotracheal intubation device, intubation stylet and hollow endotracheal tube, and are not to be construed as limiting to the presently disclosed invention.

FIG. 1a shows an embodiment of the presently disclosed intubation stylet 3 in an unflexed state. The intubation stylet 3 has a proximal part 10, a distal part 11 and a bendable part 12. The proximal part 10 extends from the handle to the bendable part 12. In this configuration, the bendable part 12 and distal part 11 are substantially straight. The lever 14 has a proximal rod element 15 and a distal rod element 16, which are pivotable at the fixed hinge 17. In FIG. 1a the intubation stylet is in an unflexed state, which means that the wire 13 follows the body of the intubation stylet.

FIG. 1b shows the intubation stylet of FIG. 1 in a flexed state. The elements are the same as in FIG. 1a, but the rod element has been turned counter clockwise around the fixed hinge 17 by pushing the distal rod element 16 in the same direction. The distal rod element 16 has reached to its end position and the device in the example cannot be bent any further.

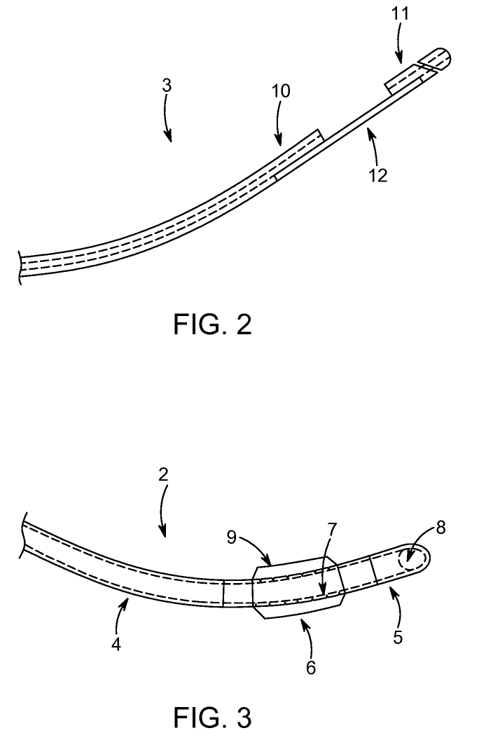

FIG. 2 shows a further embodiment of the presently disclosed intubation stylet 3 having a proximal part 10, a distal part 11 and a bendable part 12. It can be seen that the bendable part 12 is a thinner than the surrounding, abutting parts. Preferably, the bendable part 12 is made of an elastic material as described above.

FIG. 3 shows an embodiment of the presently disclosed hollow endotracheal tube 2. The tube has a proximal portion 4, a distal portion 5, a flexible portion 6 and an air hole 8 at the tip of the tube. In this example the tube also has an inflatable cuff 9. It can be seen that the surface 7 of the flexible portion 6 has grooves making it more flexible than the proximal portion 4 and the distal portion 6.

FIG. 4 shows an embodiment of the presently disclosed endotracheal intubation device having a hollow endotracheal tube 2 and an intubation stylet 3, the parts presented side by side. The tube has a proximal portion 4, a distal portion 5, an a flexible portion 6. The intubation stylet has a corresponding proximal part 10, distal part 11 and bendable part 12, the flexible portion 6 of the endotracheal tube being substantially aligned with the bendable part 12 of the intubation stylet. The lever 14 has a proximal rod element 15 and a distal rod element 16, which are pivotable at the fixed hinge 17. In this embodiment, the endotracheal tube 2 has a connection element 20, which stops against the distal edge 18 of the handle.

FIG. 5 shows the endotracheal intubation device of FIG. 4, wherein the intubation stylet is in a flexed state. In this position, the distal rod element 16 is pushed to deflect the bendable part 12. The illustration also defines the angle 19.

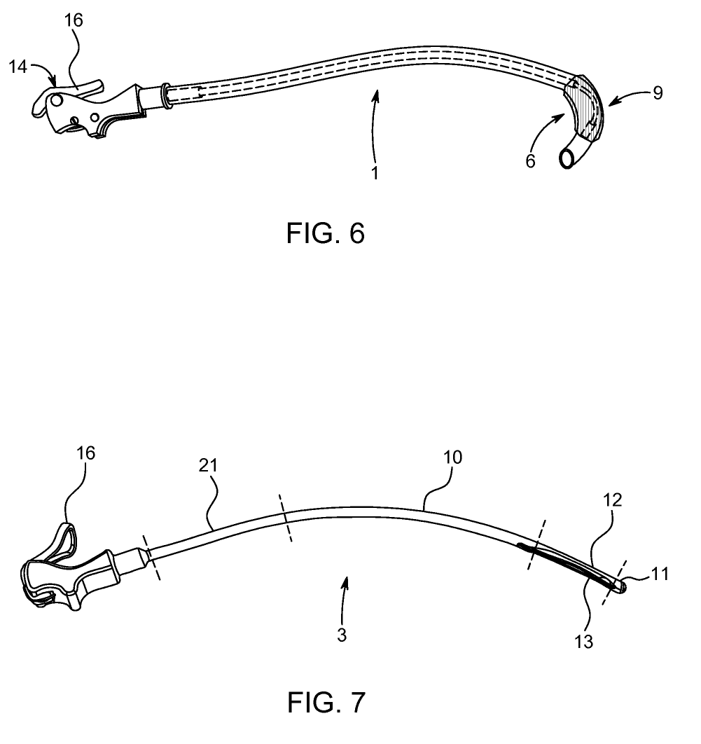

FIG. 6 shows the endotracheal intubation device 1 of FIG. 4 and FIG. 5, wherein the intubation stylet 3 is slidably inserted into an engagement position in the hollow endotracheal tube 2, the flexible portion 6 of the hollow endotracheal tube 2 being substantially aligned with the bendable part 12 of the intubation stylet.

FIG. 7 shows an embodiment of an intubation stylet (3), wherein the proximal part (10) comprises a most proximal part (21) which has a larger diameter than the rest of the intubation stylet and is substantially straight.

FIG. 8 shows an embodiment of the distal part (11) and bendable part (12) of an intubation stylet (3), wherein the bendable part (12) has a section (22) of the bendable part that is substantially flat, and the section (22) is straight in relation to the general curve of the intubation device (3) in the unflexed state. The distal part (11) has a through-hole (24) adjacent to a distal tip (23) of the intubation stylet (3), wherein the wire (13) is configured to run through the through the through-hole (24) and attach around the distal tip (23). The wire (13) is a double wire (13) in this embodiment, wherein the double wire (13) wire forms a loop (25) attached around the distal tip (23) of the intubation stylet (3).

### FURTHER DETAILS OF THE INVENTION

1. An endotracheal intubation device comprising:
   a hollow endotracheal tube having a proximal portion; a distal portion; and a flexible portion located between the proximal portion and the distal portion, the flexible portion being more flexible than the proximal portion and the distal portion; and
   an intubation stylet having a proximal part; a distal part; a bendable part located between the proximal part and the distal part, configured for slidable insertion into an engagement position in the hollow endotracheal tube; and a flexion mechanism configured to control deflection of the bendable part,
the endotracheal intubation device configured such that the flexible portion of the endotracheal tube is substantially aligned with the bendable part of the intubation stylet in said engagement position.
2. The endotracheal intubation device according to any of the preceding items, the engagement position corresponding to a first operating mode, wherein the device is configured to be inserted into the trachea of a patient.
3. The endotracheal intubation device according to any of the preceding items, wherein the bendable part is configurable in at least an unflexed state and a flexed state.
4. The endotracheal intubation device according to any of the preceding items, the proximal part and the distal part of the intubation stylet forming an angle between 50° and 179°, or between 60° and 179°, or between 70° and 179°, or between 80° and 179°, or between 60° and 160°, or between 60° and 150°, or between 60° and 140°, or between 40° and 130°, or between 60° and 120°, or between 80° and 100°, or between 70° and 110°, such as 50°, or 55°, or 60°, or 65°, or 70°, or 75°, or 80°, or 85°, or 90°, or 95°, or 100°, or 105°, or 110°, or 115°, or 120°, or 125°, or 130°, or 135°, in the flexed state.
5. The endotracheal intubation device according to any of the preceding items, wherein the bendable part of the intubation stylet is elastic.
6. The endotracheal intubation device according to any of the preceding items, wherein the bendable part of the intubation stylet comprises a coiled spring.
7. The endotracheal intubation device according to any of the preceding items, wherein the bendable part of the intubation stylet is thinner than the proximal part and the distal part of the intubation stylet.
8. The endotracheal intubation device according to any of the preceding items, the bendable part of the intubation stylet having inherent potential energy in the flexed state.
9. The endotracheal intubation device according to any of the preceding items, wherein the flexion mechanism comprises a pulling mechanism extending from a proximal end of the intubation stylet to the distal part of the intubation stylet.
10. The endotracheal intubation device according to any of the preceding items, wherein the pulling mechanism exerts a pulling force on the distal part of the intubation stylet in the flexed state, and wherein the unflexed state corresponds to a released state wherein the pulling mechanism does not pull the distal part of the intubation stylet.
11. The endotracheal intubation device according to any of items 9-10, the device configured such that the inherent potential energy in the flexed state is released when the pulling force on the distal part of the intubation stylet is released, the intubation stylet thereby returning to the unflexed state.
12. The endotracheal intubation device according to any of the preceding items, the flexion mechanism comprising a wire attached to the distal part of the intubation stylet.
13. The endotracheal intubation device according to item 12, the wire being further attached to the flexion mechanism and/or pulling mechanism.
14. The endotracheal intubation device according to any of items 12-13, the wire extending from the proximal part of the intubation stylet to the distal part of the intubation stylet, arranged through a fixed point at the intubation stylet and/or through a duct of the intubation stylet.
15. The endotracheal intubation device according to any of items 12-14, wherein the wire is a double wire.
16. The endotracheal intubation device according to any of items 12-15, the intubation stylet comprising a through-hole adjacent to a distal tip of the intubation stylet, wherein the wire is configured to run through the through the through-hole and attach around the tip.
17. The endotracheal intubation device according to any of items 15-16, wherein the double wire forms a loop, said loop attached around the distal tip of the intubation stylet.
18. The endotracheal intubation device according to any of the preceding items, further comprising a handle member, preferably a single-handed handle member, in connection with the pulling mechanism.
19. The endotracheal intubation device according to item 18, wherein the handle member is operated by pushing a pressure element connected to the wire.
20. The endotracheal intubation device according to item 19, wherein the pressure element is configured to be operated by the thumb of a user holding the handle member with the index-, middle-, ring- and little fingers, preferably by having the index-, middle-, ring- and little fingers are placed around the hollow endotracheal tube, preferably with the little finger towards the distal end of the endotracheal tube.
21. The endotracheal intubation device according to any of the preceding items, the flexion mechanism further comprising a lever, wherein a first end of the lever is connected to the wire and a second end is connected to the pressure element.
22. The endotracheal intubation device according to item 21, the lever comprising a rigid rod pivotable at a fixed hinge, wherein the lever is configured to pull the wire when the pressure element is pushed.
23. The endotracheal intubation device according to any of the preceding items, wherein the intubation stylet is curved, forming a general curve of the intubation stylet, conforming to an upper airway of the patient, and relatively stiff.
24. The endotracheal intubation device according to any of the preceding items, wherein the intubation stylet is made of a substantially rigid material, such as ceramics, ceramic composites, metal, such as aluminium or steel, or plastics, such as polycarbonate (PC) or polymethylmethacrylate (PMMA).
25. The endotracheal intubation device according to any of the preceding items, the intubation stylet having a stylet outer diameter between 1 mm and 15 mm, or between 2 mm and 15 mm, or between 1 mm and 10 mm, or between 2 mm and 10 mm, such as 1 mm, or 2 mm, or 3 mm, or 4 mm, or 5 mm, or 6 mm, or 7 mm, or 8 mm, or 9 mm, or 10 mm, or 11 mm, or 12 mm, or 13 mm, or 14 mm, or 15 mm.
26. The endotracheal intubation device according to any of the preceding items, wherein the proximal portion and the distal portion of the endotracheal tube are more rigid than the flexible portion of the endotracheal tube.
27. The endotracheal intubation device according to any of the preceding items, the flexible portion of the endotracheal tube having a thinner sidewall than the sidewalls of the proximal portion and the distal portion of the tube.
28. The endotracheal intubation device according to any of the preceding items, wherein the flexible portion of the endotracheal tube is at least 0.1 mm, or at least 0.2 mm, or at least 0.3 mm, or at least 0.4 mm, or at least 0.5 mm, or at least 0.6 mm, or at least 0.7 mm, or at least 0.8 mm, or at least 0.9 mm, or at least 1.0 mm, or at least 1.2 mm, or at least 1.4 mm, or at least 1.6 mm, or at least 1.8 mm, or at least 2.0 mm thinner than the sidewalls of the proximal portion and the distal portion of the tube.
29. The endotracheal intubation device according to any of the preceding items, the flexible portion of the endotracheal tube having grooves and/or slots and/or scratches.
30. The endotracheal intubation device according to any of the preceding items, wherein the endotracheal tube is curved, conforming to an upper airway of the patient.
31. The endotracheal intubation device according to any of the preceding items, wherein the endotracheal tube is made of plastic material, such as polyvinyl chloride (PVC), or silicone plastic.
32. The endotracheal intubation device according to any of the preceding items, wherein the length of the flexible portion is between 10 mm and 50 mm, or between 10 mm and 40 mm, or between 10 mm and 30 mm, or between 10 mm and 20 mm, or less than 50 mm, or less than 40 mm, or less than 30 mm, or less than 20 mm.
33. The endotracheal intubation device according to any of the preceding items, wherein the length of the distal part of the intubation stylet is between 10 mm and 50 mm, or between 10 mm and 40 mm, or between 10 mm and 30 mm, or between 10 mm and 20 mm, or less than 50 mm, or less than 40 mm, or less than 30 mm, or less than 20 mm.
34. The endotracheal intubation device according to any of the preceding, wherein the shape of the intubation in the unflexed configuration stylet matches the shape of the endotracheal tube.
35. The endotracheal intubation device according to any of the preceding items, wherein the device in a second operating mode is configured to be used without the intubation stylet for maintaining an open airway of the patient.
36. The endotracheal intubation device according to item 35, wherein the endotracheal tube is configured such that it is not folded during use in the second operating mode.
37. The endotracheal intubation device according to any of the preceding items, the endotracheal tube having a device outer diameter between 5 mm and 20 mm, or between 5 mm and 15 mm, or between 5 mm and 10 mm, or between 5 mm and 10 mm, such as 5 mm, or 6 mm, or 7 mm, or 8 mm, or 9 mm, or 10 mm, or 11 mm, or 12 mm, or 13 mm, or 14 mm, or 15 mm, or 16 mm, or 17 mm, or 18 mm, or 19 mm, or 20 mm.
38. The endotracheal intubation device according to any of the preceding items, the hollow endotracheal tube formed by a cylindrical sidewall having a thickness of less than 5 mm, or less than 4 mm, or less than 3 mm, or less than 2 mm, or less than 1 mm, or less than 0.5 mm, or less than 0.4 mm, or less than 0.3 mm, or less than 0.2 mm, or less than 0.1 mm.
39. The endotracheal intubation device according to any of the preceding items, the endotracheal tube having at least one primary opening at a tip at the distal portion of the endotracheal tube.
40. The endotracheal intubation device according to item 39, the endotracheal tube having at least one secondary opening at the distal portion, preferably in the cylindrical sidewall, adjacent to the tip of the endotracheal tube.
41. The endotracheal intubation device according to any of the preceding items, the device having a length of between 100 mm and 600 mm, or between 200 mm and 400 mm, or between 200 mm and 300 mm, or between 50 mm and 200 mm, or between 100 mm and 500 mm, or between 100 mm and 300 mm, or longer than 200 mm.
42. The endotracheal intubation device according to any of the preceding items, the intubation stylet further comprising a light source at the distal end.
43. The endotracheal intubation device according to item 42, further comprising a power source, such as a battery, and an electrical wire connecting the power source to the light source.
44. The endotracheal intubation device according to any of items 42-43, further comprising a power switch configured to activate and deactivate the light source.
45. The endotracheal intubation device according to item 44, wherein the power switch is configured such that the light source is enabled when the bendable part is in the flexed state and disabled when the bendable part is in the unflexed state, preferably in synchronization with the flexion mechanism, such that the pressure element closes the switch when pushed.
46. The endotracheal intubation device according to any of the preceding items, further comprising an inflatable cuff surrounding the device.
47. The endotracheal intubation device according to item 46, wherein the inflatable cuff covers the flexible portion of the endotracheal tube.
48. An intubation stylet having a proximal part; a distal part; a bendable part located between the proximal part and the distal part, configured for slidable insertion into an engagement position in the hollow endotracheal tube; and a flexion mechanism configured to control deflection of the bendable part.
49. The intubation stylet according to item 48, wherein a section of the bendable part is substantially flat.
50. The intubation stylet according to item 49, the substantially flat section having a thickness of less than 5 mm, preferably a thickness of less than 4 mm, even more preferably a thickness of less than 3 mm, most preferably a thickness of less than 2 mm.
51. The intubation stylet according to any of items 49-50, wherein the substantially flat section is straight in relation to the general curve of the intubation device in the unflexed state.
52. The intubation stylet according to any of items 49-51, wherein the substantially flat section of the bendable part has a predetermined bending direction.
53. The intubation stylet according to any of item 52, wherein the substantially flat section of the bendable part is bendable in the predetermined bending direction and substantially rigid sideways in relation to the predetermined bending direction.
54. The intubation stylet according to any of items 49-53, having a length of 5-30 mm, preferably 5-20 mm, more preferably 5-15 mm, even more preferably 8-12 mm.
55. The intubation stylet according to any of items 48-54 made of polyamide elastomer.
56. The intubation stylet according to item 48, wherein the flexion mechanism comprises a pulling mechanism extending from a proximal end of the intubation stylet to the distal part of the intubation stylet.
57. The intubation stylet according to any of items 48-56, the flexion mechanism comprising a wire attached to the distal part of the intubation stylet, the wire being further attached to the flexion mechanism and/or pulling mechanism.
58. The intubation stylet according to any of items 48-57, further comprising a handle member, preferably a single-handed handle member, in connection with the pulling mechanism.
59. The intubation stylet according to item 58, wherein the handle member is operated by pushing a pressure element connected to the wire.
60. The intubation stylet according to item 59, wherein the pressure element is configured to be operated by the thumb of a user holding the handle member with the index-, middle-, ring- and little fingers, preferably by having the index-, middle-, ring- and little fingers are placed around the intubation stylet or handle member, preferably with the little finger towards the distal end of the intubation stylet.

1. An endotracheal intubation device comprising:
a hollow endotracheal tube having a proximal portion; a distal portion; and a flexible portion located between the proximal portion and the distal portion, the flexible portion being more flexible than the proximal portion and the distal portion; and
an intubation stylet having a proximal part; a distal part; a bendable part located between the proximal part and the distal part, configured for slidable insertion into an engagement position in the hollow endotracheal tube; and
a flexion mechanism configured to control deflection of the bendable part, the endotracheal intubation device configured such that the flexible portion of the endotracheal tube is substantially aligned with the bendable part of the intubation stylet in said engagement position.

2. The endotracheal intubation device according to any of the preceding claims, wherein the proximal part of the intubation stylet comprises a most proximal part having an outer diameter at least 0.3 mm larger than the rest of the proximal part, preferably an outer diameter at least 0.5 mm larger than the rest of the proximal part, even more preferably an outer diameter at least 0.7 mm larger than the rest of the proximal part, most preferably an outer diameter at least 1.0 mm larger than the rest of the proximal part.

3. The endotracheal intubation device according to claim 2, wherein the length of the most proximal part is between 50 mm and 150 mm, preferably between 70 mm and 130 mm, even more preferably between 80 mm and 120 mm.

4. The endotracheal intubation device according to any of claims 2-3, wherein the most proximal part is substantially straight and rigid.

5. The endotracheal intubation device according to any of the preceding claims, wherein a section of the bendable part is substantially flat.

6. The endotracheal intubation device according to claim 5, the substantially flat section having a thickness of less than 5 mm, preferably a thickness of less than 4 mm, even more preferably a thickness of less than 3 mm, most preferably a thickness of less than 2 mm.

7. The endotracheal intubation device according to any of claims 5-6, wherein the substantially flat section is straight in relation to the general curve of the intubation device in the unflexed state.

8. The endotracheal intubation device according to any of claims 5-7, wherein the substantially flat section of the bendable part has a predetermined bending direction.

9. The endotracheal intubation device according to any of claims 5-8, wherein the substantially flat section of the bendable part is bendable in the predetermined bending direction and substantially rigid sideways in relation to the predetermined bending direction.

10. The endotracheal intubation device according to any of claims 5-9, the substantially flat section having a length of 5-30 mm, preferably 5-20 mm, more preferably 5-15 mm, even more preferably 8-12 mm.

11. The endotracheal intubation device according to any of the preceding claims, wherein the bendable part is configurable in at least an unflexed state and a flexed state.

12. The endotracheal intubation device according to any of the preceding claims, wherein the bendable part of the intubation stylet is elastic.

13. The endotracheal intubation device according to any of the preceding claims, wherein the bendable part of the intubation stylet comprises a coiled spring, or wherein the bendable part of the intubation stylet is thinner than the proximal part and the distal part of the intubation stylet, and/or wherein the bendable part of the intubation stylet has inherent potential energy in the flexed state.

14. The endotracheal intubation device according to any of the preceding claims, wherein the flexion mechanism comprises a pulling mechanism extending from a proximal end of the intubation stylet to the distal part of the intubation stylet, and wherein the pulling mechanism exerts a pulling force on the distal part of the intubation stylet in the flexed state, and wherein the unflexed state corresponds to a released state wherein the pulling mechanism does not pull the distal part of the intubation stylet.

15. The endotracheal intubation device according to claim 14, further comprising a handle member, preferably a single-handed handle member, in connection with the pulling mechanism.

16. The endotracheal intubation device according to any of the preceding claims, wherein the proximal portion and the distal portion of the endotracheal tube are more rigid than the flexible portion of the endotracheal tube.

17. The endotracheal intubation device according to any of the preceding claims, the flexible portion of the endotracheal tube having a thinner sidewall than the sidewalls of the proximal portion and the distal portion of the tube, and/or the flexible portion of the endotracheal tube having grooves and/or slots and/or scratches.

18. An intubation stylet having a proximal part; a distal part; a bendable part located between the proximal part and the distal part, configured for slidable insertion into an engagement position in the hollow endotracheal tube; and a flexion mechanism configured to control deflection of the bendable part.

19. The intubation stylet according to claim 18, wherein the flexion mechanism comprises a pulling mechanism extending from a proximal end of the intubation stylet to the distal part of the intubation stylet, and wherein the flexion mechanism comprises a wire attached to the distal part of the intubation stylet, the wire being further attached to the flexion mechanism and/or pulling mechanism.

20. The intubation stylet according to any of claims 18-19, wherein a section of the bendable part is substantially flat.

21. The intubation stylet according to any of claims 18-20, the substantially flat section having a thickness of less than 5 mm, preferably a thickness of less than 4 mm, even more preferably a thickness of less than 3 mm, most preferably a thickness of less than 2 mm.

22. The intubation stylet according to any of claims 18-21, wherein the substantially flat section is straight in relation to the general curve of the intubation device in the unflexed state.

23. The intubation stylet according to any of claims 18-22, wherein the substantially flat section of the bendable part has a predetermined bending direction.

24. The intubation stylet according to any of claims 18-23, wherein the substantially flat section of the bendable part is bendable in the predetermined bending direction and substantially rigid sideways in relation to the predetermined bending direction.

25. The intubation stylet according to any of claims 18-24, the substantially flat section having a length of 5-30 mm, preferably 5-20 mm, more preferably 5-15 mm, even more preferably 8-12 mm.

26. The intubation stylet according to any of claims 18-25 made of polyamide elastomer.

27. The intubation stylet according to any of claims 18-26, wherein the proximal part of the intubation stylet comprises a most proximal part having an outer diameter at least 0.3 mm larger than the rest of the proximal part, preferably an outer diameter at least 0.5 mm larger than the rest of the proximal part, even more preferably an outer diameter at least 0.7 mm larger than the rest of the proximal part, most preferably an outer diameter at least 1.0 mm larger than the rest of the proximal part.

28. The intubation stylet according to claim 27, wherein the length of the most proximal part is between 50 mm and 150 mm, preferably between 70 mm and 130 mm, even more preferably between 80 mm and 120 mm.

29. The intubation stylet according to any of claims 27-28, wherein the most proximal part is substantially straight and rigid.

30. The intubation stylet according to any of claims 18-29, wherein the flexion mechanism further comprises a wire attached to the distal part of the intubation stylet.

31. The endotracheal intubation device according to claim 30, the wire being further attached to the flexion mechanism and/or pulling mechanism.

32. The intubation stylet according to any of claims 30-31, wherein the wire is a double wire.

33. The intubation stylet according to any of claims 18-32, the intubation stylet comprising a through-hole adjacent to a distal tip of the intubation stylet, wherein the wire is configured to run through the through the through-hole and attach around the tip.

34. The intubation stylet according to any of claims 32-33, wherein the double wire forms a loop, said loop attached around the distal tip of the intubation stylet.
